(19) United States
(12) Patent Application Publication
(10) Pub. No.: US 2005/0251232 A1
(43) Pub. Date: Nov. 10, 2005

(54) Apparatus and methods for monitoring heart rate and respiration rate and for monitoring and maintaining body temperature in anesthetized mammals undergoing diagnostic or surgical procedures

(76) Inventors: Craig J. Hartley, Houston, TX (US); Sridhar Madala, Houston, TX (US); Alan D. Jones, League City, TX (US); Anilkumar K. Reddy, South Houston, TX (US)

Correspondence Address: CONLEY ROSE, P.C., P. O. BOX 3267, HOUSTON, TX, 77253-3267, US

(21) Appl. No.: 10/842,095

(22) Filed: May 10, 2004

(51) Int. Cl. A61B 5/04; A61B 5/05; A61F 7/00

(52) U.S. Cl. 607/96; 600/513; 600/547

(57) ABSTRACT

The invention relates to an apparatus for measuring the heart rate and the respiration rate of one or more anesthetized rodent while monitoring and maintaining body temperature of at least one or more anesthetized rodent during diagnostic or surgical procedures. The apparatus includes a printed circuit board having four electrodes, two of which are injection electrodes and two of which are sensor electrodes. The heart rate is monitored through known electrocardiogram techniques, however, the respiration rate is monitored by injecting an electrical current across the chest of the rodent between two of the electrodes to determine impedance of the electrical current across the chest of the rodent. The impedance is carried by a respiration signal that is then graphically displayed to measure and, thus, monitor the respiration rate. The apparatus further includes the ability to maintain and monitor the body temperature of the rodent. Methods of monitoring the respiration rate of one or more anesthetized rodents during diagnostic or surgical procedures are also disclosed.

### RIGHTS IN THE INVENTION

The invention was made with support from the United States government under grant number NIH R01 HL22512, awarded by the National Institutes of Health, and the United States government has certain rights in this invention.

### BACKGROUND OF THE INVENTION

1. Field of the Invention

This invention relates to an apparatus for use during diagnostic and surgical procedures of anesthetized mammals, such as rodents, and, in particular, to an apparatus capable of measuring and monitoring the heart rate and respiration rate of one or more anesthetized rodents. Additionally, the invention is directed to an apparatus capable of measuring and monitoring the heart rate and respiration rate of one or more anesthetized rodents while monitoring and maintaining the body temperature of the one or more rodents during the diagnostic or surgical procedure.

2. Description of Related Art

With the growth of genetic engineering, mice have become common as models of human diseases, and this in turn has stimulated the development of techniques to monitor and image the murine cardiovascular system. Invasive methods are often more quantitative, but noninvasive methods are preferred when measurements must be repeated serially on living animals during development or in response to pharmacologic or surgical interventions. Because of the small size and high heart rates in rodents and especially mice, high spatial and temporal resolutions are required to preserve signal fidelity. Monitoring of body temperature, the heart rate through an electrocardiogram (“ECG”), and the respiration rate is desired, and in most cases essential, when animals must be anesthetized for a measurement or other procedure.

The use of anesthesia, which is required for most studies in living rodents, is problematic in physiologic and pharmacologic studies because all anesthetic agents alter cardiovascular control and, thus, respiration, in some way. Anesthesia is employed to provide a consistent and controlled setting in which to study rodents; however, the type of anesthetic agent must be chosen carefully to minimize the effect on the system being studied. The rodents, however, should be monitored to ensure that the heart rate, respiration rate, and the body temperature of the rodents are constant and that the rodents are not under any stress that could interfere with the physiologic and pharmacologic studies.

To facilitate monitoring anesthetized rodents, devices have been designed to measure the ECG of the rodents so that the heart rate can be monitored. One such prior art device is the Temperature and Heart Rate Monitoring System for Mice, model number THM100, marketed and sold by Indus Instruments of Houston, Tex. This system employs a “mouse pad” which is a printed circuit board (“PCB”) approximately 8 inches by 10 inches in size to which a mouse is placed. The “mouse pad” includes ECG electrodes, surface mount resistors, and a temperature sensor arranged near the center of the board. The electrodes are placed so that the feet of the mouse are in contact with an electrode to monitor the ECG of the mouse. The resistors (approximately 50) are placed in an array under the mouse along with a temperature sensor to provide thermal support to maintain body temperature. A thermostatic controller is used to maintain board temperature at a set value between 20 and 40 degrees centigrade. The controller also contains a digital readout of the set temperature, the board temperature, or the mouse temperature via a rectal probe.

In the THM100 system, an amplifier generates an ECG signal based upon the electrical activity of the heart. Electrodes placed in contact with the body of the mouse sense the electrical activity of the heart and send the signal to the THM100 system where it is amplified to generate an ECG signal. The electrodes use standard ECG lead configurations and the THM100 includes a digital readout of heart rate. The ECG signal is suitable for display on an oscilloscope, a chart recorder, or as an input to a computerized data acquisition system. The THM100 system also includes one or more filters to separate background noise from the ECG signal so that a clearer view of the ECG signal can be obtained.

The THM100 system, however, lacks the ability to monitor the respiration rate of rodents. While other stand alone devices exist formonitoring the respiration rate, e.g., by placing a thermistor in the airway of the mouse, persons skilled in the art have sought after a more convenient and simple device for monitoring the respiration rate. Persons skilled in the art have also sought one device or system that can facilitate monitoring both heart rate and respiration rate while assisting in monitoring and maintaining body temperature.

Accordingly, prior to the development of the present invention, there has been no apparatus capable of measuring the heart rate and respiration rate of one or more anesthetized rodents while monitoring and maintaining the body temperature of the one or more of the rodents during diagnostic or surgical procedures or method of simultaneously measuring the heart rate and respiration rate of one or more anesthetized rodents while monitoring and maintaining the body temperature of the one or more of the rodents during diagnostic or surgical procedures, which: permit heart rate and respiration rate to be monitored simultaneously from a single device using the same set of electrodes while monitoring and maintaining body temperature; provide easy set up for monitoring the heart and respiration rates; reduces the number of sensors or electrodes need to measure both heart rate and respiration rate; and increase the accessibility to the test subject during diagnostic or surgical procedures by reducing the number of probes, sensors and other instruments required to monitor heart and respiration rate and control body temperature. Therefore, the art has sought an apparatus capable of measuring the heart rate and respiration rate of one or more anesthetized rodents while monitoring and maintaining the body temperature of the one or more of the rodents during diagnostic or surgical procedures and methods of simultaneously measuring the heart rate and respiration rate of one or more anesthetized rodents while monitoring and maintaining the body temperature of the one or more of the rodents during diagnostic or surgical procedures, which: permit heart rate and respiration rate to be monitored simultaneously from a single device using the same set of electrodes while monitoring and maintaining body temperature; provide easy set up for monitoring the heart and respiration rates; reduces the number of sensors or electrodes need to measure both heart rate and respiration rate; and increase the accessibility to the test subject during diagnostic or surgical procedures by reducing the number of probes, sensors and other instruments required to monitor heart and respiration rate and control body temperature. It is believed that the present invention will achieve these objectives and overcome the disadvantages of other similar apparatuses and methods, but the results or effects are still dependent upon the skill and training of the operators and surgeons.

### SUMMARY OF INVENTION

In accordance with the invention the foregoing advantages have been achieved through the present apparatus for monitoring a heart rate and a respiration rate of at least one rodent having a heart generating a heart voltage, the apparatus comprising: a printed circuit board having at least two injection electrodes and at least one sensor electrode, the at least two injection electrodes being in electrical communication with an electrical current source to create an electrical circuit passing from one of the at least two injection electrodes, through the rodent, to another of the at least two injection electrodes, the electrical circuit having a known voltage, and the at least one sensor electrode being adapted to measure a change in the known voltage of the electrical circuit and being adapted to measure a heart voltage generated by the heart of the rodent; and a control box having a heart rate monitor and a respiration rate monitor, the control box being in electrical communication with a power source, the respiration rate monitor having the electrical current source, wherein at least one of the at least one sensor electrodes is in electrical communication with the respiration rate monitor for determining the respiration rate of the at least one rodent based upon changes in the known voltage of the electrical circuit, and wherein at least one of the at least one sensor electrodes is in electrical communication with the heart rate monitor for determining the heart rate of the at least one rodent based upon changes in the heart voltage.

A further feature of the apparatus is that the electrical circuit may have a frequency in the range from 1 to 100 kHz. Another feature of the apparatus is that the electrical circuit may have a frequency of 50 kHz. An additional feature of the apparatus is that the printed circuit board may have at least two injection electrodes and at least two sensor electrodes. Still another feature of the apparatus is that the at least two injection electrodes and at least two sensor electrodes may be disposed in a rectangular arrangement. A further feature of the apparatus is that the control box may further comprise a body temperature controller and at least one resistor disposed along the printed circuit board, the body temperature controller having a temperature electrical current source for creating a temperature electrical circuit, and the body temperature controller being in electrical communication with the at least one resistor whereby the temperature electrical current is passed from the temperature electric current source to the at least one resistor. Another feature of the apparatus is that the apparatus may further comprise a thermometer in electrical communication with the body temperature controller. An additional feature of the apparatus is that the respiration rate monitor may include a respiration signal amplifier and at least one respiration electrical signal filter, and the heart rate monitor may include a heart rate signal amplifier and at least one heart rate electrical signal filter. Still another feature of the apparatus is that the control box may further include a temperature controller having at least one temperature control and a temperature electrical current source for creating a temperature electrical circuit, and the printed circuit board includes at least one resistor in electrical communication with the temperature electrical current source. A further feature of the apparatus is that the temperature controller may further include a thermometer in electrical communication with the temperature controller. Another feature of the apparatus is that the thermometer may be a rectal thermometer. An additional feature of the apparatus is that the temperature controller may include a temperature display, the heart rate monitor includes a heart rate display and the respiration rate monitor includes a respiration rate display. Still another feature of the apparatus is that each of the at least one sensor electrodes may be adapted to measure a change in the known voltage of the electrical circuit and being adapted to measure the heart voltage. A further feature of the apparatus is that the sensor electrode adapted to measure a change in the known voltage of the electrical circuit may be different from the sensor electrode adapted to measure the heart voltage. Another feature of the apparatus is that the at least one rodent may be at least one mouse.

In accordance with the invention the foregoing advantages have also been achieved through the present method of monitoring a respiration rate of a rodent comprising the steps of: contacting at a first injection electrode to a rodent having a chest; contacting a second injection electrode to the rodent; contacting a sensor electrode to the rodent; passing an electrical current having a known voltage from the first injection electrode, across the chest of the rodent, to the second electrode; measuring by the sensor electrode a change in the voltage of the electrical current after it passes from the first injection electrode, across the chest of the rodent, and to the second electrode; and determining the respiration rate of the rodent based upon the change in the known voltage of the electrical current as a function of time after it passes from the first injection electrode, across the chest of the rodent, and to the second electrode.

A further feature of the method is that the at least one rodent may be at least one mouse.

In accordance with the invention the foregoing advantages have also been achieved through the present method of monitoring a respiration rate and a heart rate of a rodent comprising the steps of: contacting at a first injection electrode to a rodent having a heart generating a heart voltage and a chest; contacting a second injection electrode to the rodent; contacting a sensor electrode to the rodent; passing an electrical current having a known voltage from the first injection electrode, across the chest of the rodent, to the second electrode; measuring by the sensor electrode a change in the known voltage of the electrical current after it passes from the first injection electrode, across the chest of the rodent, and to the second electrode; determining the respiration rate of the rodent based upon the change in the known voltage of the electrical current as a function of time after it passes from the first injection electrode, across the chest of the rodent, and to the second electrode; measuring by the sensor electrode a change in the heart voltage generated by the heart; and determining the heart rate of the rodent based upon the change in the voltage as a function of time.

A further feature of the method is that the first sensor electrode and the second sensor electrode may be the same electrode. Another feature of the method is that the first sensor electrode and the second sensor electrode may be different electrodes. An additional feature of the method is that the at least one rodent may be at least one mouse.

The apparatuses capable of measuring the heart rate and respiration rate of one or more anesthetized rodents while monitoring and maintaining the body temperature of the one or more of the rodents during diagnostic or surgical procedures and methods of simultaneously measuring the heart rate and respiration rate of one or more anesthetized rodents while monitoring and maintaining the body temperature of the one or more of the rodents during diagnostic or surgical procedures of the present invention have the advantages of: permitting heart rate and respiration rate to be monitored simultaneously from a single device using the same set of electrodes while monitoring and maintaining body temperature; providing easy set up for monitoring the heart and respiration rates; reducing the number of sensors or electrodes need to measure both heart rate and respiration rate; and increasing the accessibility to the test subject during diagnostic or surgical procedures by reducing the number of probes, sensors and other instruments required to monitor heart and respiration rate and control body temperature. As mentioned above, it is believed that the present invention will achieve these objectives and overcome the disadvantages of other other similar apparatuses and methods in the field of the invention, but the results or effects are still dependent upon the skill and training of the operators and surgeons.

### BRIEF DESCRIPTION OF DRAWINGS

FIG. 1A is a perspective view of one embodiment of the control box of the monitoring device for monitoring heart rate and respiration rate and for monitoring and maintaining the body temperature of anesthetized mammals of the present invention.

FIG. 1B is a perspective view of one embodiment of the platform of the monitoring device for monitoring heart rate and respiration rate and for monitoring and maintaining the body temperature of anesthetized mammals of the present invention.

FIG. 2 is a top view of one embodiment of the printed circuit board of the embodiment of the invention shown in FIG. with an anesthetized mouse placed on the printed circuit board.

FIG. 3A is an electrocardiogram generated by one embodiment of the apparatus for monitoring heart rate and respiration rate of the present invention in which no lowpass filter is utilized.

FIG. 3B is another electrocardiogram generated by one embodiment of the apparatus for monitoring heart rate and respiration rate of the present invention in which a 1 kHz lowpass filter is utilized.

FIG. 3C is an additional electrocardiogram generated by one embodiment of the apparatus for monitoring heart rate and respiration rate of the present invention in which a 100 Hz lowpass filter is utilized.

FIG. 3D is still another electrocardiogram generated by one embodiment of the apparatus for monitoring heart rate and respiration rate of the present invention in which a 30 Hz lowpass filter is utilized.

FIG. 4 is a graph of a respiration signal generated by one embodiment of the apparatus for monitoring respiration rate of the invention.

While the invention will be described in connection with the preferred embodiment, it will be understood that it is not intended to limit the invention to that embodiment. On the contrary, it is intended to cover all alternatives, modifications, and equivalents, as may be included within the spirit and scope of the invention as defined by the appended claims.

### DETAILED DESCRIPTION AND SPECIFIC EMBODIMENTS

Broadly, the present invention is directed to methods of monitoring the respiration rate of a rodent based upon the impedance value calculated across the rodent's body. The impedance value is determined by injecting current through an injection electrode in contact with the rodent's body. A second electrode, referred to as a measurement electrode, also in contact with the rodent's body, receives the current after it has passed across the chest of the rodent. The change in voltage across the rodent's body generates a respiration signal that reflects the impedance value and that is sent back to the control box where it can be amplified or viewed as a graph, similar to an ECG, on an oscilloscope or other monitoring device. The respiration rate can then be determined by measuring the amount of time between peaks on the graph, or between troughs on the graph, e.g., breaths per minute. The peaks on the graph are the points of time at which the lungs have their maximum volume of air (inhalation) and the troughs on the graph are the points of time at which the lungs have their minimum volume of air (exhalation). Therefore, the respiration signal can be monitored.

In another aspect, the present invention is directed to an apparatus capable of monitoring the heart rate, the respiration rate, and, preferably, the body temperature of an anesthetized rodent. The apparatus includes a platform, e.g., a printed circuit board having at least three electrodes. The apparatus uses electrical current injected across at least two electrodes in contact with the rodent to determine the respiration rate. At least one electrode is utilized to measure the electrical activity of the heart to determine the heart rate and to simultaneously measure the change in voltage of the electrical current passing between the other two electrodes across the body of the rodent. Preferably, four electrodes are utilized with one electrode is in contact with each of the paws of the rodent. The change in the voltage across the rodent's body generates a respiration signal as discussed above that can be viewed on an oscilloscope or other monitoring device. As such, the respiration rate can be determined and, thus, monitored based upon this respiration signal. Additionally, the ECG of the rodent can be monitored by using the electrodes to sense the electrical activity of the heart and send an ECG signal to the control box in the same manner as other ECG devices. The present apparatus, however, is capable of receiving both the respiration signal and the ECG signal from the same set of electrodes. In preferred embodiments, the control box includes one or more filters to facilitate separation of the respiration signal, the ECG signal, and background noise or interference contained within the signal.

The apparatus also preferably includes a thermometer for measuring the body temperature of the rodent and a heater built into the platform on which the rodent is placed. The thermometer is contacted with the rodent, e.g., rectally or through skin contact, and is placed in electrical communication with a body temperature display so that the body temperature of the rodent can be monitored. The heater is formed by one or more resistors placed in the platform through which electricity is passed. As the electricity passes through the resistors, heat is generated and transferred from the platform to the rodent's body. In this way, the rodent's body remains warm, and optimally keep at the rodent's homeostatic temperature. A board temperature sensor is also preferably utilized to monitor the temperature of the platform.

The present invention will be discussed in greater detail with respect to mice; however, it is to be understood that the present invention is capable of monitoring respiration rate, and of monitoring heart rate, body temperature and respiration rate, of all types of mammals including rodents.

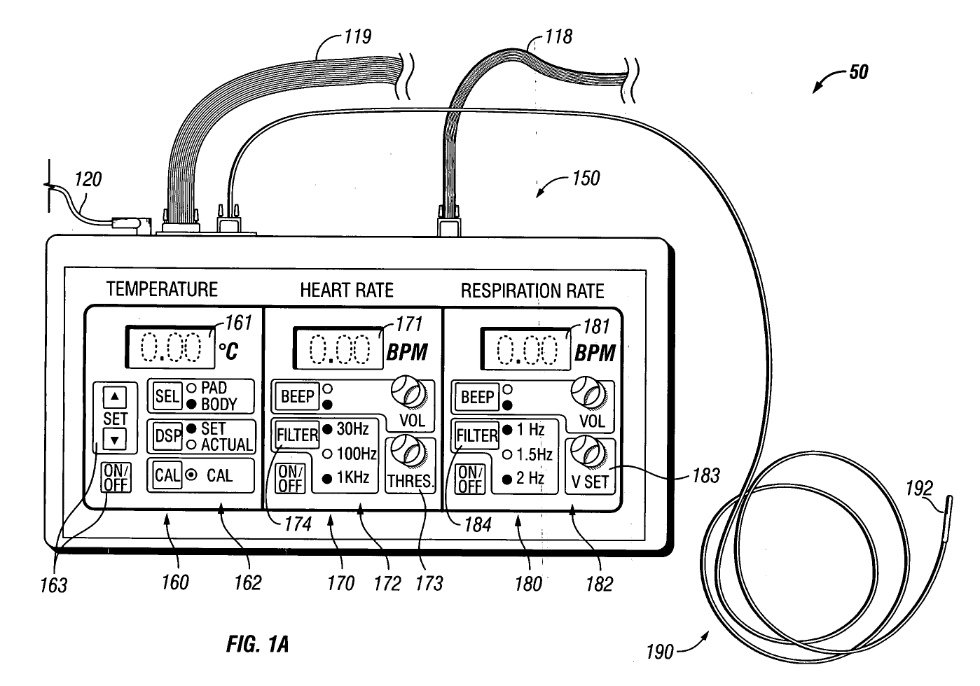

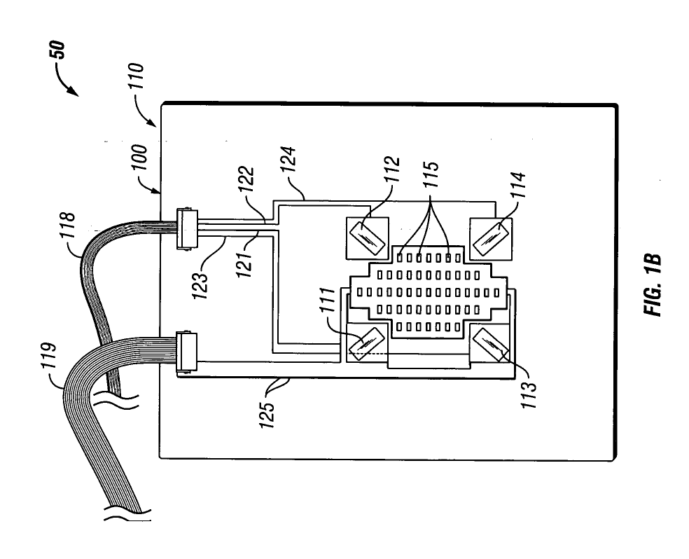

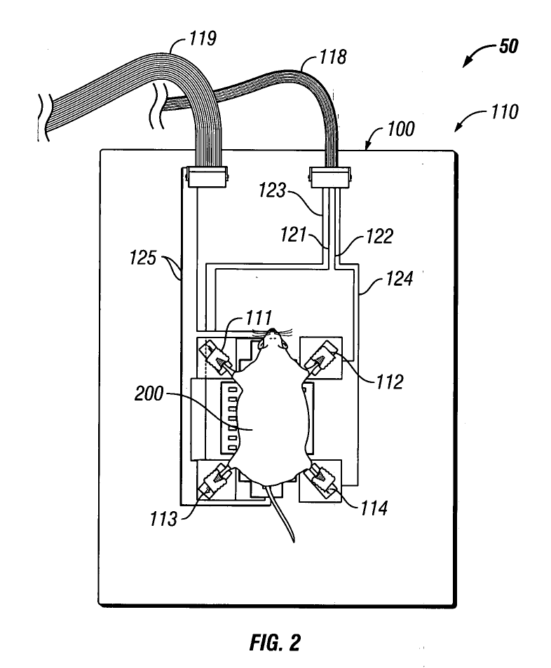

Referring now to FIGS. 1-2, in one embodiment of the present invention, the apparatus, or monitoring device 50, includes platform 100 which, as shown in FIG. 1, is printed circuit board 110. Printed circuit board 110 includes at least two electrodes. As shown in FIG. 1, printed circuit board includes four electrodes, 111, 112, 113, 114. Each of the electrodes 111, 112, 113, 114 is in electrical communication with control box 150 through electrical leads 121, 122, 123, 124, respectively.

As shown in FIG. 1, four electrodes, 110, 111, 112, 113 are disposed on printed circuit board 110 in a rectangular arrangement. Interestingly, this rectangular arrangement permits measurement of the respiration rate despite the conventional wisdom that impedance measurements through such an arrangement have not been shown to be very sensitive.

Printed circuit board 110 also includes one or more resistors 115 in electrical communication with control box 150 through electrical leads 125. Resistors 115 provide heat due to an electrical current being passed from an electrical power source through each of the resistors 115. The generated heat, in turn, is transferred to the mouse's body that is in contact with each of the resistors 115. Preferably, printed circuit board 110 includes a plurality of resistors 115 arranged in a shape complementary to the shape of the mouse 200 (FIG. 2) so that heat generated by the resistors 115 will be transferred to a larger surface area of the mouse's body. Because the resistors 115 are in electrical communication with an adjustable power source as part of temperature controller 160, the electrical current passing through the resistors 115 can be regulated, i.e., increased or decreased. By increasing the electrical current through the resistors, the amount of heat generated by the resistors 115 is increased. Therefore, the amount of heat available to be transferred to the mouse can likewise be increased as desired or necessary to maintain the mouse's homeostatic temperature. Alternatively, by decreasing the electrical current through the resistors, the amount of heat generated by the resistors 115 is decreased. Therefore, the amount of heat available to be transferred to the mouse can likewise be decreased as desired or necessary to maintain the mouse's homeostatic temperature. An example of a suitable printed circuit board with resistors is represented by the THM100 system of Indus Instruments discussed above.

Control box 150 includes power cord 120 for electrical communication with a power source (not shown), e.g., 120 volt AC outlet, temperature controller 160, heart rate monitor 170, respiration rate monitor 180, respiration/heart rate cable 118, and temperature cable 119. Temperature controller 160 includes temperature display 161 and temperature controls 162. Temperature controls 162 include adjustable power source 163 for generating an electrical current, the flow of which to resistors 115 can be increased or decreased. Such adjustable power sources are known to persons skilled in the art.

Temperature controller 160 is also in electrical communication with resistors 115 through electrical leads 125 so that the temperature of resistors 115 disposed along printed circuit board 110 and the temperature of printed circuit board 110 can be monitored. In a preferred embodiment, temperature controller 160 is also in electrical communication with thermometer 190 which is in contact with mouse 200 so that the body temperature of mouse 200 can be determined and displayed and, thus, monitored, on body temperature display 161. Thermometer 190 may be a rectal thermometer 192, as shown in FIG. 1, or a skin contact thermometer (not shown). An example of a suitable temperature controller and thermometer apparatus is represented by the THM100 system of Indus Instruments discussed above.

Heart rate monitor 170 includes heart rate display 171 and heart rate controls 172. Heart rate monitor 170 also includes heart rate signal amplifier 173, known to persons skilled in the art, to increase or decrease the amplitude of the heart rate signal for better detection and monitoring.

In certain embodiment, heart rate monitor 170 also includes one or more electrical signal filters 174 built into heart rate monitor 170 to separate the heart rate signal from the respiration signal created by high frequency impedance signal discussed below in greater detail, and any background signals, i.e., interference or noise. Electrical signal filters 174 are known to persons skilled in the art. Preferably, heart rate monitor 170 includes two or more different filters, e.g., a 1 kHz lowpass filter, a 100 Hz lowpass filter, and a 30 Hz lowpass filter, so that the degree of separation of the heart rate signal can be modified to increase the clarity or sensitivity of the heart rate signal. With proper selection of frequencies, waveforms, impedances, detection methods, and filters; it is possible to measure the heart rate and the respiration rate using the same electrodes at the same time.

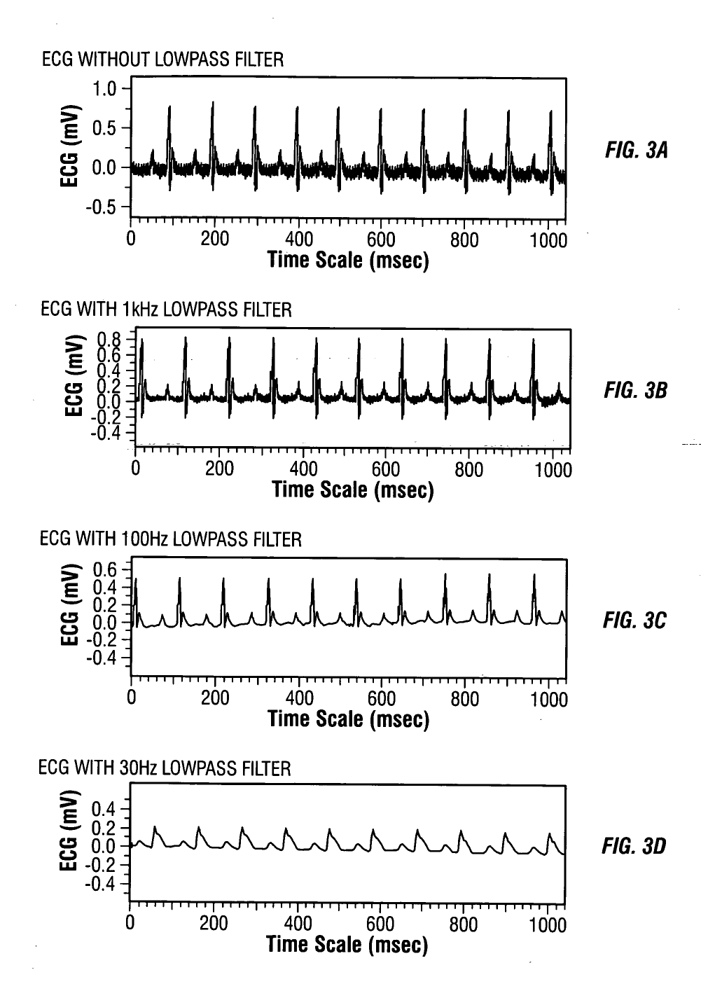

Because the heart generates an electric signal which is present all over the body including the limbs, the heart rate is measured as the voltage difference between any two of the electrodes (each connected to a limb) on the board. The electrodes that measure the voltage are referred to herein as “sensor electrodes.” The particular sensor electrodes used may be selected by one of the heart rate controls 172 located on heart rate monitor 170, and in some configurations, signals from several sensor electrode pairs can be recorded and displayed at the same time. The heart rate signals are on the order of a few millivolts and contain frequencies from zero to 1 kHz with the fundamental frequency of 1-10 Hz being the heart rate signal. An electrocardiograph of the heart rate signal can be them be created and displayed in the same manner as shown in FIGS. 3A-3D.

An example of a suitable heart rate monitor is represented by the THM100 system of Indus Instruments discussed above.

As opposed to the heart, the lungs do not generate electrical signals during breathing. There is, however, a change in electrical impedance across the chest during breathing because air has a higher impedance than blood and other body tissues. The method for measuring respiration is based upon thoracic impedance which changes as a function of lung volume; the more air in the lung, the higher the electrical impedance across the chest or thorax.

Impedance is measured by injecting a known electrical current between two electrodes and then measuring the resulting voltage as a function of time. Because of the arrangement of electrodes, voltage can be measured using a separate pair of electrodes (the sensor electrodes) than the ones used to inject the current (the injection electrodes). A few μA of alternating (“AC”) current (“I”) at a frequency of 1-100 kHz is injected across the injection electrodes and the resulting voltage (“E”) across the sensor electrodes is measured. In accordance with Ohm's law (E=IR), the measured voltage is the product of the injected current and the electrical resistance (“R”). After band-pass filtering at the driving frequency, the AC voltage signal is rectified and then low-pass filtered at about 100 Hz to produce a voltage proportional to impedance and, thus, the respiration rate can be monitored.

As illustrated in FIGS. 1-2, respiration rate monitor 180 is in electrical communication with each of electrodes 111, 112, 113, 114 and is utilized to generate and measure the respiration signal. Respiration rate monitor 180 includes a respiration rate display 181, an electrical current source, or power source 183, for generating an electrical current at known voltages, the flow of which to one or more electrodes 111, 112, 113, 114 can be increased or decreased. Such electrical current sources 183 are preferably adjustable and are known to persons skilled in the art.

The electrical current is transmitted from the respiration rate monitor 180 to at least one electrode 111, 112, 113, 114 through the body of mouse 200, to another electrode 111, 112, 113, 114 and back to respiration rate monitor 180 to complete an electrical circuit. For example, in one embodiment, the electrical current is transmitted from the respiration rate monitor 180 to electrode 113, across the body of mouse 200, to electrode 112, and then back to respiration rate monitor 180 to complete the electrical circuit. In this embodiment, electrodes 113, 112 are “injection electrodes” because they are the electrodes through with the electrical current is passed to complete the electrical circuit; and electrodes 111, 114 are “sensor electrodes” because they are the electrodes that detect the change in voltage as the electrical current passes through the body of mouse 200. In this embodiment, electrodes 111, 114 also detect the electrical activity of the heart so that the heart rate can be measured and monitored.

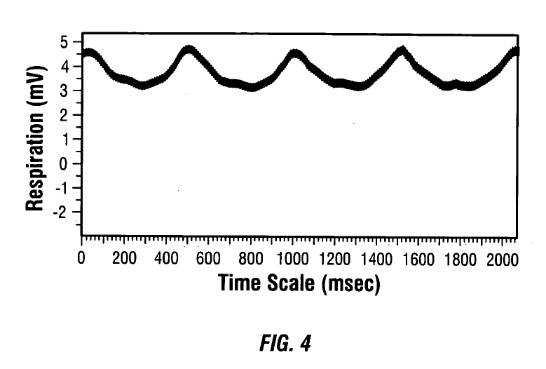

With respect to the respiration rate, the change in the voltage as measured by sensor electrodes 111, 114 may then be plotted on a graph (see e.g., FIG. 4) and the amount of time between waves, i.e., the change in amplitude, can be measured to determine the respiration rate in breaths per minute. The respiration rate can then be displayed on respiration rate display 181.

As mentioned above, the respiration signal is derived from the change in thoracic impedance measured using a high frequency carrier on the injection electrodes. Preferably, electrodes on opposite corners of the mouse, e.g., electrodes 111, 114 or 112, 113, are used to inject current at a frequency in the 10-50 KHz range (“injection electrodes”), and the two other electrodes, e.g., 112, 113 or 111, 114, respectively, are used to measure the resulting voltage, i.e., the “sensor electrodes.” The amplitude modulation of the resulting respiration signal measured by the sensor electrodes is related to impedance which can be recorded and displayed similar to the heart rate signal. Therefore, monitoring device 50 is capable of measuring the heart rate and the respiration rate with the same sensor electrodes.

To avoid interference with the heart rate signal, current is injected and voltage is measured at a high frequency, e.g., between 1-100 kHz, and preferably at about 50 kHz. High frequencies are preferably used in monitoring the respiration rate because higher currents can be used without adverse biological consequences, and 60 Hz and ECG artifacts, or noise, can be eliminated by filtering using electrical signal filters similar to those discussed above with respect to heart rate monitor 170. Therefore, respiration rate monitor 180 preferably includes one or more electrical signal filter 184 to separate the respiration signal from the heart rate signal and any background noise or interference. Electrical signal filters 184 are known to persons skilled in the art. It is contemplated that respiration rate monitor 180 will include two or more different filters, e.g., 0.1 Hz, 1 Hz, 1.5 Hz, and 2.0 Hz filters, so that the degree of separation of the respiration signal can be modified to increase the clarity or sensitivity of the respiration signal.

Additionally, the electrical current injected across the mouse for monitoring the respiration rate is preferably capacitively coupled using a high impedance driving circuit to minimize the effect on the ECG current paths within the body of the animal. Also, four electrodes, two injection electrodes and two sensor electrodes, are preferably used to eliminate the effect of electrode impedance in the measurements.

Respiration of mouse 200 modulates the amplitude of the 50 kHz voltage signal, and a tuned detector similar to that used in an AM radio is used to recover the low frequency (zero to 20 Hz) respiration signal. Electrical signal filters 184 are preferably used to tune the receiver to 50 kHz to eliminate the low frequencies from the respiration signal before detection. Similar filters are also used within the to eliminate any high frequency signals. In this way, both the heart rate signal and the respiration rate signal can be measured at the same time from the same electrodes.

From the heart rate signal and the respiration signal standard methods can be used to determine heart rate (at 1-10 Hz) and respiratory rate (at 0.1-2 Hz) by measuring the primary or fundamental frequency of each signal at these different frequencies.

As discussed above, preferably, four electrodes 111, 112, 113, 114 are utilized with two electrodes, e.g., 113, 112 being injection electrodes, i.e., receiving the electrical current from respiration rate monitor 180, and two electrodes, e.g., 111, 114 being the sensor electrodes, i.e., measuring the electrical current passing through the body of mouse 200 or emanating from the heart of mouse 200, and sending both the respiration signal and the heart rate signal to respiration rate monitor 180 and heart rate monitor 170, respectively.

In certain embodiments, respiration rate monitor 180 includes a respiration signal amplifier 183, also known to persons skilled in the art, to increase the amplitude of the respiration signal for better detection and monitoring. It is to be understood that respiration signal amplifier 183 and heart rate signal amplifier 173 may be the same component of control box 150. It is also to be understood that respiration signal amplifier 183 may include one or more electrical signal filters 184.

It is to be understood that the injection electrodes maybe any of the electrodes 111, 112, 113, 114. It is also to be understood that the sensor electrodes measuring the electrical current passing through the body of mouse 200 or emanating from the heart of mouse 200 may be any of the electrodes 111, 112, 113, 114. Preferably, two of electrodes 111, 112, 113, 114 are injection electrodes and two of electrodes 111, 112, 113, 114 are sensor electrodes.

By incorporating a heating element such as resistors 115 discussed above, together with electrodes 111, 112, 113, 114 on printed circuit board 110 with connections to a temperature controller 160, and heart rate monitor 170, and respiration monitor 180, handling of rodents during surgery, imaging, and other biological experiments is significantly simplified.

It is to be understood that the invention is not limited to the exact details of construction, operation, exact materials, or embodiments shown and described, as obvious modifications and equivalents will be apparent to one skilled in the art. For example, while the invention has been discussed in connection with mice, the invention may be utilized with other rodents and other small animals. Moreover, the adjustable power source of the temperature controller may be the same adjustable power source utilized by the respiration rate monitor. Additionally, even though the present inventions have been discussed herein with respect to rodents, and in particular, mice, it is contemplated that the respiration rate of any mammal can be determined using the methods and apparatuses of the inventions. Further, a personal computer can be used to monitor, display, and record the body temperature, the heart rate, and the respiration rate. Accordingly, the invention is therefore to be limited only by the scope of the appended claims.

1. An apparatus for monitoring a heart rate and a respiration rate of at least one rodent having a heart generating a heart voltage, the apparatus comprising:
a printed circuit board having at least two injection electrodes and at least one sensor electrode,
the at least two injection electrodes being in electrical communication with an electrical current source to create an electrical circuit passing from one of the at least two injection electrodes, through the rodent, to another of the at least two injection electrodes, the electrical circuit having a known voltage, and
the at least one sensor electrode being adapted to measure a change in the known voltage of the electrical circuit and being adapted to measure a heart voltage generated by the heart of the rodent; and
a control box having a heart rate monitor and a respiration rate monitor, the control box being in electrical communication with a power source, the respiration rate monitor having the electrical current source,
wherein at least one of the at least one sensor electrodes is in electrical communication with the respiration rate monitor for determining the respiration rate of the at least one rodent based upon changes in the known voltage of the electrical circuit, and
wherein at least one of the at least one sensor electrodes is in electrical communication with the heart rate monitor for determining the heart rate of the at least one rodent based upon changes in the heart voltage.

2. The apparatus of claim 1, wherein the electrical circuit has a frequency in the range from 1 kHz to 100 kHz.

3. The apparatus of claim 1, wherein the electrical circuit has a frequency of 50 kHz.

4. The apparatus of claim 1, wherein the printed circuit board has at least two injection electrodes and at least two sensor electrodes.

5. The apparatus of claim 4, wherein the at least two injection electrodes and at least two sensor electrodes are disposed in a rectangular arrangement.

6. The apparatus of claim 1, wherein the control box further comprises a body temperature controller and at least one resistor disposed along the printed circuit board, the body temperature controller having a temperature electrical current source for creating a temperature electrical circuit, and the body temperature controller being in electrical communication with the at least one resistor whereby the temperature electrical current is passed from the temperature electric current source to the at least one resistor.

7. The apparatus of claim 6, further comprising a thermometer in electrical communication with the body temperature controller.

8. The apparatus of claim 1, wherein the respiration rate monitor includes a respiration signal amplifier and at least one respiration electrical signal filter, and
wherein the heart rate monitor includes a heart rate signal amplifier and at least one heart rate electrical signal filter.

9. The apparatus of claim 8, wherein the control box further includes a temperature controller having at least one temperature control and a temperature electrical current source for creating a temperature electrical circuit, and the printed circuit board includes at least one resistor in electrical communication with the temperature electrical current source.

10. The apparatus of claim 9, wherein the temperature controller further includes a thermometer in electrical communication with the temperature controller.

11. The apparatus of claim 10, wherein the thermometer is a rectal thermometer.

12. The apparatus of claim 11, wherein the temperature controller includes a temperature display, the heart rate monitor includes a heart rate display and the respiration rate monitor includes a respiration rate display.

13. The apparatus of claim 1, wherein each of the at least one sensor electrodes is adapted to measure a change in the known voltage of the electrical circuit and being adapted to measure the heart voltage.

14. The apparatus of claim 1, wherein the sensor electrode adapted to measure a change in the known voltage of the electrical circuit is different from the sensor electrode adapted to measure the heart voltage.

15. The apparatus of claim 1, wherein the at least one rodent is at least one mouse.

16. A method of monitoring a respiration rate of a rodent comprising the steps of:
contacting at a first injection electrode to a rodent having a chest;
contacting a second injection electrode to the rodent;
contacting a sensor electrode to the rodent;
passing an electrical current having a known voltage from the first injection electrode, across the chest of the rodent, to the second electrode;
measuring by the sensor electrode a change in the voltage of the electrical current after it passes from the first injection electrode, across the chest of the rodent, and to the second electrode; and
determining the respiration rate of the rodent based upon the change in the known voltage of the electrical current as a function of time after it passes from the first injection electrode, across the chest of the rodent, and to the second electrode.

17. The method of claim 16, wherein the at least one rodent is at least one mouse.

18. A method of monitoring a respiration rate and a heart rate of a rodent comprising the steps of:
contacting at a first injection electrode to a rodent having a heart generating a heart voltage and a chest;
contacting a second injection electrode to the rodent;
contacting a sensor electrode to the rodent;
passing an electrical current having a known voltage from the first injection electrode, across the chest of the rodent, to the second electrode;
measuring by the sensor electrode a change in the known voltage of the electrical current after it passes from the first injection electrode, across the chest of the rodent, and to the second electrode;
determining the respiration rate of the rodent based upon the change in the known voltage of the electrical current as a function of time after it passes from the first injection electrode, across the chest of the rodent, and to the second electrode;
measuring by the sensor electrode a change in the heart voltage generated by the heart; and
determining the heart rate of the rodent based upon the change in the voltage as a function of time.

19. The method of claim 18, wherein the first sensor electrode and the second sensor electrode are the same electrode.

20. The method of claim 18, wherein the first sensor electrode and the second sensor electrode are different electrodes.

21. The method of claim 18, wherein the at least one rodent is at least one mouse.

22. The method of claim 18, wherein the electrical current has a frequency in the range from 1 kHz to 100 kHz.
